(19) United States
(12) Patent Application Publication
(10) Pub. No.: US 2026/0216411 A1
(43) Pub. Date: Jul. 30, 2026

(54) CONTROL DEVICE OR CLOSED-LOOP CONTROL DEVICE FOR A BLOOD TREATMENT APPARATUS FOR REGULATING THE CLEARANCE DURING DIALYSIS

(71) Applicant: Fresenius Medical Care Deutschland GmbH, Bad Homburg (DE)

(72) Inventor: Andreas Maierhofer, Bad Homburg (DE)

(21) Appl. No.: 19/143,122

(22) PCT Filed: Jan. 10, 2024
(86) PCT No.: PCT/EP2024/050445

§ 371 Date: Jun. 25, 2025

(30) Foreign Application Priority Data
Jan. 12, 2023 (DE) 10 2023 100 665.7

(51) Int. Cl. A61M 1/16

(52) U.S. Cl. CPC A61M 1/1603; A61M 2205/3327; A61M 2205/3334; A61M 2205/50

(57) ABSTRACT

The present disclosure relates to a control device or closed-loop control device configured to control or regulate an extracorporeal blood treatment apparatus, when being connected thereto in signal communication. The blood treatment apparatus comprises a dialyzer being divided by a semipermeable membrane into a blood chamber and a dialysis liquid chamber. The control device or closed-loop control device is further configured to set a dialysate flow for an extracorporeal blood treatment by the extracorporeal blood treatment apparatus, wherein the control device or closed-loop control device is configured such that the dialysate flow for the dialyzer of the blood treatment apparatus is set such that over a plurality of successive time intervals within the blood treatment session, a first dialysate flow and a second dialysate flow alternate with each other, wherein the first dialysate flow is higher than the second dialysate flow.

### CROSS-REFERENCE TO RELATED APPLICATIONS

The present application is the national stage entry of International Patent Application No. PCT/EP2024/050445, filed Jan. 10, 2024, and claims priority to Application No. DE 102023100665.7, filed in the Federal Republic of Germany on Jan. 12, 2023, the contents of which are hereby incorporated by reference.

### TECHNICAL FIELD

The present disclosure relates to a control device or closed-loop control device for controlling or controlling in a closed-loop manner a blood treatment apparatus. The present disclosure further relates to a digital storage medium, to a computer program product, and to a computer program.

### BACKGROUND

In hemodialysis performed by a blood treatment apparatus, the efficiency of blood purification by the blood filter, or dialyzer, may be indicated by the degree of clearance. The clearance is set mainly by the properties of the membrane of the blood filter, parameters of the blood filter, as well as the blood flow in the blood chamber and the dialysate flow in the dialysis liquid chamber of the blood filter.

The blood purification that benefits the patient is reduced on the patient side by recirculation which describes the proportion of blood already purified in the blood filter, which proportion ends up in the arterial line without equilibration with the total body reservoir having taken place after being returned to the patient's vascular system via a venous line. Reasons for this may include cardiopulmonary recirculation or direct recirculation in the vascular access or central venous catheter.

### SUMMARY

When reference is made herein to a “patient”, this refers to a person whose blood requires treatment. This notation does not imply any information as to the gender or other characteristics of this person.

In hemodialysis (HD), the substance exchange or substance removal of uremic toxins occurs primarily by diffusion. This is set by the concentration gradient between blood and dialysis liquid and the clearance. This mechanism can be effective for small molecules such as urea. Since the velocity of diffusion decreases with increasing molecule size, diffusion is less effective for larger molecules. In this case, convective removal via ultrafiltration is more effective. This method is used in hemofiltration (HF).

Hemodialysis and hemofiltration may be combined for the hemodiafiltration (HDF). By selecting the operating parameters in HDF, the substance-specific proportion of diffusion and convection may be adjusted within certain limits.

The total dialysis dose achieved in a dialysis treatment is described by the parameter Kt/V. In this, K is the urea clearance in the blood filter in units of [ml/min], t is the treatment time in minutes [min], and V is the urea distribution volume or the patient's body water in units of [ml]. All statements made herein about urea also apply to other uremic toxins.

In general, it is advantageous to select the dialysis parameters such that the highest possible dialysis dose is achieved. However, undesirable side effects may also occur due to the substance exchange in the blood filter.

This includes, above all, the dialysis disequilibrium syndrome. This occurs when the removal of mainly low-molecular uremic toxins, which may be present in relatively high concentrations (approx. 1-100 mmol/L), takes place too quickly. One mechanism of harm here is that the concentration of toxins in different body compartments being in exchange with each other has equalized in the time before dialysis. Since removal by dialysis occurs directly from only one of the compartments (extracellular compartment), the resulting difference in concentration with other compartments, e.g., the intracellular compartment and the brain, causes osmotically-induced volume shifts. These may lead to neurological symptoms. The urea concentration can be responsible for this.

This mainly affects patients who are newly treated with chronic hemodialysis or goes with dialyses after a longer dialysis-free period, e.g., after missed dialysis sessions. However, comparable effects are also observed in acute dialysis after acute renal failure or also in the treatment of intoxication, e.g., in connection with the removal of alcohol after alcohol intoxication.

It may be an aspect of the present disclosure to provide a control device or closed-loop control device for controlling or controlling in a closed-loop matter a blood treatment apparatus, and a further blood treatment apparatus.

The control device or closed-loop control device described herein can be configured for controlling or controlling in a closed-loop manner an extracorporeal blood treatment apparatus when connected thereto in signal communication. The blood treatment apparatus serves for extracorporeally treating the blood of a patient in a blood treatment session and, for this purpose, comprises, or is connected to, a blood filter (herein also: dialyzer) which is divided into a blood chamber and a dialysis liquid chamber by a semipermeable membrane. During the blood treatment, blood flows through the blood chamber with a predetermined blood flow, while during treatment (fresh) dialysis liquid flows into the dialysis liquid chamber with a predetermined dialysate flow, and (used) dialysate flows out of the dialysis liquid chamber. This flow through the dialysis liquid chamber is referred to herein as the dialysate flow. In this, herein, dialysate flow may refer to the fluid flow flowing into and/or flowing out of the dialysis liquid chamber.

The control device or closed-loop control device described herein is further configured for setting or specifying a dialysate flow for an extracorporeal blood treatment generated by or with the extracorporeal blood treatment apparatus. The control device or closed-loop control device is configured such that the dialysate flow flowing through the dialyzer of the blood treatment apparatus is set or specified such that a first dialysate flow and a second dialysate flow alternate with each other over a plurality of successive time intervals (e.g., identical or different, variable or constant time intervals) within the blood treatment session under consideration. In this, the first dialysate flow is higher than the second dialysate flow, alternatively it is lower.

The control device or closed-loop control device may be programmed as described above. Alternatively, a calculation unit and/or evaluation unit is configured as described, e.g., for setting or specifying the dialysate flow. The calculation unit and/or evaluation unit may be a part of the control device or closed-loop control device, or may be separate therefrom. In the latter case, the control device or closed-loop control device may be, or prepared to be, in signal communication with the calculation unit and/or evaluation unit.

The blood treatment apparatus can comprise, be connected to, or be configured to be in signal communication to a control device or closed-loop control device as described herein.

A digital, e.g., non-volatile storage medium, e.g., in the form of a machine readable carrier, e.g., in the form of a diskette, CD, DVD EPROM, FRAM (Ferroelectric RAM) or SSD (Solid-State-Drive), e.g., with electronically or optically readable control signals may interact with a computer system such that a conventional control device or closed-loop control device of a blood treatment apparatus is reprogrammed into a control device or closed-loop control device as described herein.

A computer program product as described herein can comprise a volatile or transient program code or one stored on a machine-readable carrier or a signal wave, with which a conventional control device or closed-loop control device of a blood treatment apparatus is reprogrammed into a control device or closed-loop control device as described herein when the computer program product runs on a computer. Computer program product can be for example understood as a computer program stored on a carrier, an embedded system being a comprehensive system with a computer program (e.g., an electronic device with a computer program), a network of computer implemented computer programs (e.g., client/server-system, a cloud computing system etc.), or a computer on which a computer program is loaded, runs, is stored, is being executed or developed.

The term “machine readable carrier” as used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

A computer program described herein can comprise a program code with which a conventional control device or closed-loop control device of a blood treatment apparatus is reprogrammed into a control device or closed-loop control device as described herein.

Embodiments described herein may comprise one, several or all of the features mentioned below in any combination.

In all of the aforementioned and following statements, the use of the expression “may be” or “may have” and so on, is to be understood synonymously with “preferably is” or “preferably has”, and so on respectively, and is intended to illustrate embodiments described herein.

Whenever numerical words are mentioned herein, the person skilled in the art shall recognize or understand them as indications of numerical lower limits. Hence, unless this leads to a contradiction evident for the person skilled in the art, the person skilled in the art shall comprehend for example “one” (or “a/an”) as encompassing “at least one”. This understanding is also equally encompassed by the present disclosure as the interpretation that a numerical word, for example, “one” (or “a/an”) may alternatively mean “exactly one”, wherever this is evidently technically possible in the view of the person skilled in the art. Both of these understandings are encompassed by the present disclosure and apply herein to all used numerical words.

The person skilled in the art shall understand spatial information like, e.g., “top”, “bottom”, “left” or “right”, whenever they are herein mentioned, as a spatial indication with reference to the alignment in the figures appended hereto and/or during use. “Bottom” is closer to the geocenter or to the lower edge of the figure than “top”.

Whenever an embodiment is mentioned herein, it represents an exemplary embodiment according to the present disclosure which is not to be understood as limiting.

When it is disclosed herein that the subject-matter according to the present disclosure comprises one or several features in a certain embodiment, it is also respectively disclosed herein that the subject-matter according to the present disclosure does, in other embodiments, likewise according to the present disclosure, explicitly not comprise this or these features, for example, in the sense of a disclaimer. Therefore, for every embodiment mentioned herein it applies that the converse embodiment, e.g., formulated as negation, is also disclosed.

When method steps are mentioned herein, then the control device or closed-loop control device as described herein is in several embodiments configured in order to execute, in any combination, one, several or all of these method steps, e.g., when these are automatically executable steps, or in order to accordingly control corresponding apparatuses/devices which can correspond with their names to the designation of the respective method step (for example, “determining” as a method step and “apparatus for determining/determining device” for the apparatus/device) and which may likewise be part of the apparatus(es) according to the present disclosure or connected thereto in signal communication.

When programmed or configured is mentioned herein, then these terms may in some embodiments be interchangeable.

When a signal communication or communication connection between two components is mentioned herein, this may be understood to mean a connection that exits during use. It may also be understood that a preparation for such a (wired, wireless or otherwise implemented) signal communication exists, for example by coupling both components, for instance by pairing, etc.

Pairing is a process that takes place in connection with computer networks in order to establish an initial link between computer units for the purpose of communication. An example of this is the establishing of a Bluetooth connection, by which various devices (e.g., smartphone, headphones) are connected to each other. Pairing is sometimes also referred to as bonding.

The control device or closed-loop control device may prompt all or substantially all of the method steps disclosed herein to be executed. The method disclosed herein may be executed substantially or entirely by the control device or closed-loop control device. It may be partially executed by the control device or closed-loop control device, and in some embodiments those steps may be executed by the control device or closed-loop control device which do not require, or relate to, human intervention and/or an initialization. The control device may be used purely as a control device or also as a closed-loop control device.

In several embodiments, the control device or closed-loop control device lies in or on the blood treatment apparatus, for instance together with other components or devices of the blood treatment apparatus in a common housing of the blood treatment apparatus.

In some embodiments, the control device or closed-loop control device comprises, or is in signal communication with, or is prepared for signal communication to a measuring device for measuring at least one characteristic value for the clearance. In these embodiments, the control device or closed-loop control device is configured to determine the level or value of the first dialysate flow and/or the second dialysate flow based on the at least one characteristic value for the clearance. Alternatively, it is configured to determine a target value for the first dialysate flow and/or the second dialysate flow based on the at least one characteristic value.

In several embodiments, the control device or closed-loop control device comprises instead of the measurement device an estimation device for estimating the clearance. This can be done easily, for example if the dialyzer parameter KOA and the set or measured flows are known. This case is of economic importance for “low-cost apparatuses” or acute apparatuses without clearance measurement.

In some embodiments, a communication device is provided via which the user may make inputs to determine the clearance. Such inputs may be or include, a mean clearance or target clearance K target (related to the entire treatment session or individual time intervals) being pre-set and intended to be achieved by the treatment session, the parameter of the blood filter, its effective value, the patient's shunt flow, its cardiac output, the recirculation known from previous treatments, and results of clearance determinations from previous treatments of the currently treated patient or other patients.

In several embodiments, the control device or closed-loop control device is configured to respectively set or specify, for a time interval under consideration out of the plurality of time intervals, a first time fraction during which within the considered time interval dialysis liquid is conveyed with the first dialysate flow through the dialysis liquid chamber. In addition or alternatively, it is configured to set or specify a second time fraction during which within the considered time interval dialysis liquid is conveyed through the dialysis liquid chamber with the second dialysate flow.

In some embodiments, the first dialysate flow may in each time interval be the same, e.g., constant. It may alternatively be different in several or in all of the time intervals of the blood treatment session.

In several embodiments, the second dialysate flow may be the same, e.g., constant, in each time interval. It may alternatively be different in several or in all of the time intervals of the blood treatment session.

In several embodiments, the control device or closed-loop control device specifies or sets a pattern in which the first dialysate flow and the second dialysate flow alternately flow through the dialyzer. In some embodiments, the control device or closed-loop control device specifies or sets the points of time of, e.g., periodic successive, switching processes between the first dialysate flow and the second dialysate flow.

In some embodiments, the first dialysate flow may be a maximum dialysate flow being adjustable at the blood treatment apparatus. Alternatively or additionally, the second dialysate flow through the dialysis liquid chamber may be zero.

Such a dialysate flow through the dialysis liquid chamber equal to zero may be achieved, for example, by switching off the dialysis liquid production or its conveyance, or by directing the dialysis liquid past the dialysis liquid chamber, for instance by a bypass line.

In several embodiments of the control device or closed-loop control device, the first time fraction during which within the time interval under consideration dialysis liquid is conveyed with the first dialysate flow through the dialysis liquid chamber and the second time fraction during which within the time interval under consideration dialysis liquid is conveyed with the second dialysate flow through the dialysis liquid chamber of the blood filter may be set such that, during the considered time interval, the blood conveyed through the blood chamber of the blood filter is purified when passing through the blood chamber with a pre-set mean clearance.

In some embodiments, the control device or closed-loop control device is configured to be in signal communication, during use, with one or several sensors of a measurement device and/or to determine the clearance from the signals communicated to it by the sensors or otherwise.

In several embodiments the control device or closed-loop control device is configured in order to change the first time fraction during which within the time interval under consideration dialysis liquid is conveyed with the first dialysate flow through the dialysis liquid chamber and/or the second time fraction during which within the time interval under consideration dialysis liquid is conveyed with the first dialysate flow through the dialysis liquid chamber based on the values, determined, e.g., measured, by the sensors, such that the pre-set mean clearance is achieved within the considered time interval.

In some embodiments, the control device or closed-loop control device comprises or is, or is prepared to be, in signal communication with a communication device, by which one or several value(s) may be entered, for example values for time fractions, time durations or time intervals, the value of the mean clearance, the clearances in the first time fraction or in the second time fraction or similar. These values can be suitable or provided for setting or defining the dialysate flows required for the extracorporeal blood treatment.

In some embodiments, the blood treatment apparatus according to the present disclosure comprises or is connected to a dialyzer. The dialyzer is in turn divided by a semipermeable membrane into a blood chamber, and a dialysis liquid chamber, wherein blood may flow through the blood chamber with a predetermined blood flow and dialysis liquid may flow through the dialysis liquid chamber with a predetermined dialysate flow.

In several embodiments the blood treatment apparatus is embodied as a dialysis apparatus, hemodialysis apparatus, hemofiltration apparatus or hemodiafiltration apparatus, e.g., as an apparatus for the acute kidney replacement therapy, the chronic kidney replacement therapy or for the continuous kidney replacement therapy (CKRT).

In some embodiments, a change in the clearance is not caused by a change in blood flow. However, if a change in the blood flow causes a change in the clearance, the control device or closed-loop control device is not configured to selectively effect the clearance by changing the blood flow. In other words, in these embodiments, the blood flow does not play a role in the calculation of flows by the control device or closed-loop control device in order to achieve the desired or pre-set mean clearance, e.g., not for the respective time interval.

In several embodiments, the control device or closed-loop control device is not configured to determine a difference between a clearance based on values measured by the sensors and the pre-set mean clearance, e.g., for the respective time interval. In some embodiments, it is not configured to prompt an increase or decrease of the dialysate flow based on the calculation of said difference.

Some or all embodiments according to the present disclosure may have one, several or all of the advantages mentioned supra and/or below.

One advantage of the present methods, systems, and devices may be that it is possible to reduce the clearance K without the hitherto existing disadvantages, or with reducing said existing disadvantages.

For dialysis operation, minimum flow rates are usually necessary due to technical reasons. Should the clearance K be influenced by the present methods, systems, and devices, changes in the blood flow done by mostly manual adjustments by the user or by complex adjustments on the manufacturer-side of the monitoring system of the blood treatment apparatus, which are impractical, may be advantageously omitted.

Another possibility for reducing the clearance K is the arrangement in the parallel current principle, e.g., blood and dialysis liquid flow through the blood filter in the same direction, whereby a reduction of approx. 20% may be achieved. During the treatment of the patient, however, it is then practically impossible to switch to the more efficient countercurrent principle, in which blood and dialysis liquid flow through the blood filter in opposite directions. A further advantage of the present methods, systems, and devices may be that a reduction in clearance K may also be achieved in the more efficient countercurrent principle.

A further advantage of the present methods, systems, and devices may include advantageously avoiding hemofiltration (without diffusive exchange), in which low convective flows and thus low clearances K may be set (in the case of devices typically equipped for hemofiltration). The method of hemofiltration without diffusive exchange is available on only a small fraction of the devices on the market because of the increased technical effort and the costs associated therewith. Hence, by the present methods, systems, and devices, both the increased technical effort can be avoided and the significantly higher costs associated therewith can be saved. In addition, the availability of the present methods, systems, and devices is significantly increased compared to hemofiltration (without diffusive exchange).

Blood treatment apparatuses with fixed flow rates are less complex and less expensive. For this reason, the dialysate flow in the blood treatment apparatuses of the prior art is for technical reasons also not arbitrarily adjustable, but is often set by or to only a few fixed flow rates, e.g., 300 and 500 ml/min, or certain minimum flow rates. By the present methods, systems, and devices, this problem of lack of controllability in a closed-loop manner on the dialysate side in reducing the clearance K may also be advantageously avoided.

Another advantage of the present methods, systems, and devices may be that it makes it possible to add drugs in the arterial branch of the system, instead of in the venous branch as in common practice. Since this allows the addition of drugs to be carried out on the negative pressure side of the extracorporeal system, devices are possible by which the withdrawal from a reservoir containing the drug to be added may be carried out solely by the arterial negative pressure of the extracorporeal system. This may help to save time and reduce the need for personnel. Furthermore, an infusion pump of the blood treatment apparatus (or an external pump for this purpose) may be advantageously omitted.

All the advantages achievable with the method steps mentioned herein may in certain embodiments according to the present disclosure be achieved to an undiminished extent also with the devices according to the present disclosure.

### BRIEF DESCRIPTION OF THE FIGURES

In the following, the present disclosure will be merely exemplarily described with reference to the accompanying figures. In them, same reference numerals denote identical or similar components. In the figures, the following applies:

FIG. 1 shows schematically simplified a fluid line diagram of a blood treatment apparatus in a first embodiment;

FIG. 2 shows a blood treatment apparatus with a control device or closed-loop control device in a first embodiment during use;

FIG. 3 shows the time course of a clearance-controlled change of the dialysate flow during the treatment of a patient by a blood treatment apparatus, comprising a control device or closed-loop control device, in a further embodiment;

FIG. 4 shows a kinetic 2-pool model for substance exchange between body compartments IC and EC.

FIG. 5a shows a simulation of the material concentration course assuming an intercompartment clearance of 800 ml/min;

FIG. 5b shows a simulation of the material concentration course assuming an intercompartment clearance of 100 ml/min;

FIG. 6a shows a possible concentration course on the dialysate side downstream of the blood filter without liquid removal by ultrafiltration; and

FIG. 6b shows a possible concentration course on the dialysate side upstream of the blood filter with liquid removal by ultrafiltration.

### DETAILED DESCRIPTION

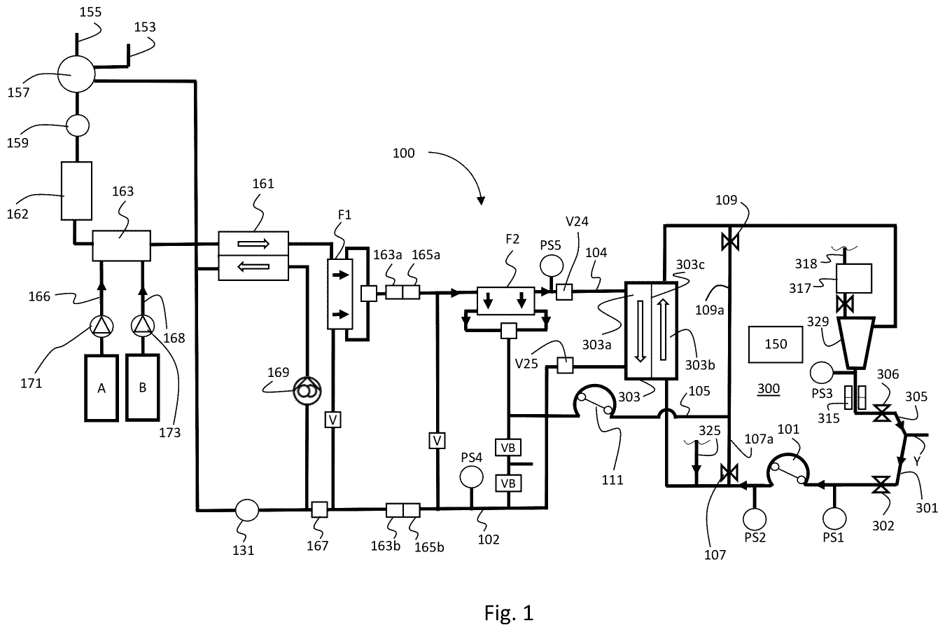

FIG. 1 shows a fluid line diagram of a blood treatment apparatus 100 in a first embodiment. The blood treatment apparatus is, in FIG. 1, represented only through single components being partially highly schematically simplified.

The blood treatment apparatus 100, which is shown in an at least partially equipped ready-for-use state, is connected to an extracorporeal blood circuit 300, which can be connected to the vascular system of the patient, not shown, for treatment using double-needle access, or by using e.g., an additional Y-connector (reference numeral Y), as shown in FIG. 1, wherein said extracorporeal blood circuit 300 is optionally not part of the blood treatment apparatus 100; however it is part thereof in other embodiments. The blood circuit 300 may be present, optionally in sections thereof, in or on a blood cassette.

Pumps, actuators and/or valves in the area of the blood circuit 300 are connected in signal communication to the blood treatment apparatus 100 or to a control device or closed-loop control device 150, encompassed by it, in the event that controlling or controlling in a closed-loop manner is required.

The blood circuit 300 comprises (or is connected to) an arterial patient tube clamp 302 and an arterial connection needle (not shown in FIG. 1) of an arterial section or of an arterial patient line, blood withdrawal line or first line 301.

The blood circuit 300 also comprises (or is connected to) a venous patient tube clamp 306 and a venous connection needle (not shown in FIG. 1) of a venous section, a venous patient line, a blood return line or a second line 305.

A blood pump 101 is provided in or at the first line 301, an optional substitute fluid pump 111 is connected to, e.g., a dialysis liquid inlet line 104 for conveying fresh dialysis liquid, which is filtered (substitute fluid) in a filtering step (filter F2).

An optional substitute fluid line 105 may be fluidically connected, for example, to a dialysis liquid inlet line 104. Using the substitute fluid pump 111, substitute fluid may be introduced by pre-dilution, via an optional pre-dilution valve 107, or by post-dilution, via an optional post-dilution valve 109, via optional associated lines 107a or 109a into line sections, for example into the arterial line section 301 or into the venous line section 305 (here between a blood chamber 303b of a blood filter 303 and a venous air separation chamber or venous blood trap 329) of the blood circuit 300.

The blood filter 303 comprises the blood chamber 303b connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, which guides dialysate, e.g., spent dialysis liquid, leading away from the dialysis liquid chamber 303a. Suitable connectors on the dialysis liquid inlet line 104 or on the dialysate outlet line 102 on the one hand, and on the dialysate port of the blood filter 303 on the other hand, which may be connected, e.g., releasably, to each other, serve for this purpose.

Dialysis liquid chamber 303a and blood chamber 303b are separated from each other by a mostly semipermeable membrane 303c. It represents the partition between the blood side with the extracorporeal blood circuit 300 and the machine side with the dialysis liquid circuit or dialysate circuit, which is shown in FIG. 1 to the left of the membrane 303c.

The arrangement of FIG. 1 comprises an optional detector 315 for detecting air and/or blood. The arrangement of FIG. 1 optionally further comprises one or two pressure sensors PS1 (upstream of the blood pump 101) and PS2 (downstream of the blood pump 101, it measures the pressure upstream of the blood filter 303 (“pre-hemofilter”)) at the sites shown in FIG. 1. Further pressure sensors may be provided, such as pressure sensor PS3 downstream of venous bubble trap 329.

An optional single-needle chamber 317 is used in FIG. 1 as a buffer and/or compensating reservoir in a single-needle procedure in which the patient is connected to the extracorporeal blood circuit 300 using only one of the two blood lines 301, 305.

An addition site 325 for Heparin or for another, e.g., local, anticoagulants may optionally be provided.

On the left in FIG. 1, an optional mixing device 163 is shown, which provides a pre-set mixture for the respective solution from the containers A (for A-concentrate via concentrate supply 166) and B (for B-concentrate via concentrate supply 168) for use by the blood treatment apparatus 100. The solution contains water from the water source 155 (on-line, e.g., as reverse osmosis water or from bags), which water is heated, e.g., in the heating device 162.

An optional pump 171, which can be referred to as concentrate pump or sodium pump, is fluidically connected to the mixing device 163 and to a source of sodium, such as the container A, and/or conveys out of it. An optional pump 173 associated with container B, such as for bicarbonate, can be seen.

Furthermore, FIG. 1 shows a drainage 153 for the effluent. An optional heat exchanger 157 and an optional first flow pump 159, which is suitable for degassing, complete the arrangement shown.

The optional pressure sensor PS4 may be provided downstream of the blood filter 303 on the water side, but can be upstream of an optional ultrafiltration pump 131 in the dialysate outlet line 102 for measuring the filtrate pressure or membrane pressure of the blood filter 303.

The ultrafiltration pump 131 provides a means for accurately removing, from the balanced circuit, a volume of liquid being specified by the user and/or by the control device or closed-loop control device 150.

Blood leaving the blood filter 303 flows through an optional venous bubble trap 329, which may comprise a deaeration device 318 and may be in fluid communication with the pressure sensor PS3.

The exemplary arrangement shown in FIG. 1 comprises the control device or closed-loop control device 150. The latter may be in a wired or wireless signal connection with any of the components mentioned herein—e.g., with the blood pump 101—in order to control or regulate the blood treatment apparatus 100.

By using the device for on-line mixing of the dialysis liquid, a variation of its sodium content, controlled by the control device or closed-loop control device 150, is possible within certain limits. For this purpose, e.g., the measured values determined by the conductivity sensors 163a, 163b may be taken into account. Should an adjustment of the sodium content of the dialysis liquid (sodium concentration) or of the substitute fluid turn out to be necessary or desired, this can be done by adjusting the conveying rate of the sodium pump 171.

In addition, the treatment apparatus 100 comprises devices for conveying fresh dialysis liquid and dialysate. An optional first valve V24 may be provided between the first flow pump 159 and the blood filter 303, which first valve V24 opens or closes the inflow towards the blood filter 303 at the inlet side. A second, optional flow pump 169 which conveys dialysate to the drainage 153 is provided, e.g., downstream of the blood filter 303. A second valve V25 may be provided between the blood filter 303 and the second flow pump 169, which second valve V25 opens or closes the outflow at the outlet side.

Furthermore, the blood treatment apparatus 100 optionally comprises a device 161 for balancing the flow flowing into and out of the dialyzer 303 on the machine side. The device 161 for balancing can be arranged in a line section between the first flow pump 159 and the second flow pump 169.

Sensors such as the optional conductivity sensors 163a, 163b serve to determine the conductivity, which in some embodiments is temperature-compensated, as well as the fluid flow upstream and downstream of the dialyzer 303.

Optional temperature sensors 165a, 165b may be provided as one or a plurality thereof. Temperature values supplied by them may be used to determine a temperature-compensated conductivity.

A leakage sensor 167 is optionally provided. It may be provided also at a different position.

Further flow pumps in addition to or alternatively to e.g., the one with the reference numeral 169 may also be provided.

A number of optional valves are each denoted with V in FIG. 1; by-pass valves with VB.

A pressure sensor P25 for measuring the pressure in the dialysis liquid inlet line 104 may be provided.

The control device or closed-loop control device 150 determines in several embodiments the electrolyte balance and/or liquid balance based on the measured values from the aforementioned optional sensors.

Filters F1 and F2 can be provided being connected in series.

Even when using non-pure water, the filter F1 exemplarily serves herein to generate sufficiently pure dialysis liquid by the mixing device 163, which then flows through the blood filter 303, e.g., using the countercurrent principle.

The filter F2 exemplarily serves herein to generate sterile or sufficiently filtered substitute fluid from the sufficiently pure dialysis liquid leaving the first filter F1, by filtering, e.g., pyrogenic substances. This substitute fluid may then be safely added to the extracorporeally flowing blood of the patient and thus ultimately to the patient's body.

Although the blood treatment apparatus 100 is optionally shown in FIG. 1 as an apparatus for hemo(dia) filtration, nevertheless hemodialysis apparatuses are also covered by the present disclosure, even though not specifically represented in a figure.

The arrows shown in FIG. 1 generally indicate the flow direction in FIG. 1.

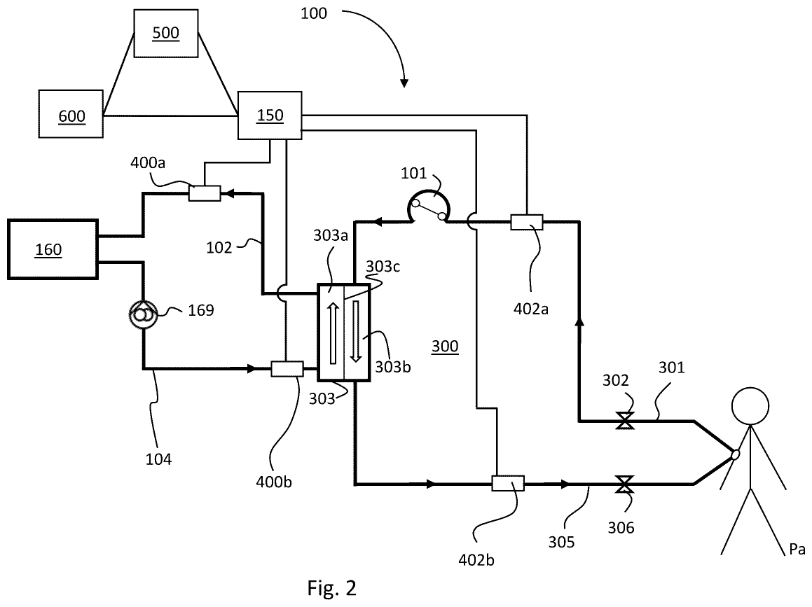

FIG. 2 shows a blood treatment apparatus 100 with a control device or closed-loop control device 150 in a first embodiment during use.

Using a blood pump 101, blood is withdrawn through the vascular access of the patient Pa via an arterial (first) connection needle and delivered to a blood filter or dialyzer 303 via a first line 301. The blood pump 101 may be part of a blood treatment apparatus 100 or integrated into a disposable item. Any suitable methods may be used for conveying, for example, conveying by peristaltic pumps or by impeller pumps.

The blood filter 303 may be any unit for performing hemodialysis (HD), hemofiltration (HF), hemodiafiltration (HDF), or a combination thereof.

The apparatus comprises means for dialysis liquid preparation, for example as set forth with respect to FIG. 1, wherein the fresh dialysis liquid is conveyed by a suitable controllable flow pump 169 via the dialysis liquid inlet line 104 to the blood filter 303, e.g., into its dialysis liquid chamber 303a. In this, the flow pump 169 may also be considered and also referred to as a “loading pump”, via which the dialysis liquid flow may be adjusted based on the rotational speed of the flow pump 169.

The balancing device 161 cyclically supplies over a suitable switching of valves fresh dialysis liquid into the dialyzer 303 via the filter F1 and the dialyzer valve V24, herein in short also valve.

The balancing device 161 alternately ensures that the volume of the dialysis liquid that flows into the dialyzer 303 is equal to the volume that flows back via the balancing chambers of the balancing device 161. A pressure sensor between the flow pump 169 and the balancing device 161 detects a filled balancing chamber because of the pressure increase, and changes the valve switching (open/closed) accordingly.

The flow pump 169 ensures that dialysate coming from the dialyzer 303 is supplied into the balancing chambers of the balancing device 161.

For example, the means for preparing dialysis liquid comprises itself means for changing the composition of the dialysis liquid by changing the mixing ratio of the components involved in the on-line generation of dialysis liquid, for example as explained in FIG. 1.

The used dialysate is returned or returns via a dialysate outlet line 102 to an optional means for reprocessing dialysate 160. Optionally, the second flow pump 169 and/or other means for liquid withdrawal by ultrafiltration may serve for this purpose, which may be provided in the dialysis liquid inlet line 104 or the dialysate outlet line 102. Likewise, means for, e.g., continuously measuring the dialysate flow may be included in one of these lines 102, 104.

In some embodiments, the blood treatment apparatus 100 may further comprise means for branching off a partial flow for the purpose of substitution from the total dialysate as specified by the control device or closed-loop control device 150, so that an HF treatment or HDF treatment may be carried out using a balancing device 161 (see FIG. 1) in predilution or postdilution. The partial flow is exemplarily brought from the flow coming from the balancing device 161 and the filter F1 via the filter F2 into the subsequent fluid line and is introduced into the extracorporeal blood circuit 300 by the substitute fluid pump 111 via the predilution line 107a and/or the postdilution line 109a.

In the example of FIG. 2, the blood treatment apparatus 100 comprises two blood-side sensors 402a, 402b and two dialysate-side sensors 400a, 400b, which are respectively arranged upstream and downstream of the blood filter 303.

These sensors 400a, 400b, 402a, 402b are suitable and provided for setting the concentration of a substance, e.g., sodium, contained in the blood or in the dialysate, respectively, or a value correlating therewith, by any method of contact or non-contact measurement. For example, this may involve ion-selective electrodes, conductivity sensors or spectroscopic devices for measurement in the infrared, visible or UV range. At least one sensor 400b, 402b, which is arranged downstream of the blood filter 303 on the blood-side or dialysate-side, is hereby used.

The sensors of the same or different types may be used such that, by combining, or based on, the measured values of the different sensors or substance concentrations or values correlating with substance concentrations, it becomes possible to calculate using the control device or closed-loop control device 150 the clearance K (or dialysance) of one or more substances or groups of substances. This may include methods that do not interfere with the course of treatment, as well as methods that carry out variations on the dialysis liquid composition or flows for the purpose of determining the clearance.

The connection of the sensors 400a, 400b, 402a, 402b to the measuring points on the blood-side or dialysate-side may be permanently installed in the blood treatment apparatus 100. Alternatively, the sensors may be positioned or inserted completely or partially only during preparation of the blood treatment apparatus 100. For example, the sensors 400a, 400b, 402a, 402b may be part of disposables on the blood-side and/or the dialysate-side.

The connection between the sensors 400a, 400b, 402a, 402b and the control device or closed-loop control device 150 may be wired or wireless.

In some embodiments, the control device or closed-loop control device 150 is programmed or configured to calculate a clearance K, a dialysis dose Kt, and Kt/V, respectively, based on the measured values of the sensors 400a, 400b, 402a, 402b, and other measured values and control variables of the blood treatment apparatus 100, e.g., based on blood-side and dialysate-side flows.

Values averaged over a time interval may be used instead of or in addition to the currently determined values. If the sensors 400a, 400b, 402a, 402b are conductivity sensors or ion-selective measuring sensors, the control device or closed-loop control device 150 may calculate, e.g., by using the known formulas or formulas mentioned herein, the blood-side concentrations of the substances of interest resulting from both the direct calculation as well as with the aid of a kinetic model for the exchange over different body compartments, e.g., during periods without dialysate flow.

The values relating to clearance K, dialysis dose Kt or substance concentration may be output to the user or an external observer via a display device 500 and/or a communication device 600.

Furthermore, it may be possible to specify a specification for the desired temporal course of the clearance K using the display device 500 and the communication device 600. This specification is implemented using the blood treatment apparatus 100.

Further, it may be possible, using the display device 500 and the communication device 600, to input or to receive parameters that allow an approximate value of the clearance K to be calculated for the various dialysate flows Q_{d} used. These include, e.g., the dialyzer parameter K₀A or its effective value, the patient's shunt flow, their cardiopulmonary recirculation, their cardiac output, the recirculation known from previous treatments, and the results of clearance measurements from previous treatments of the currently treated patient or other patients.

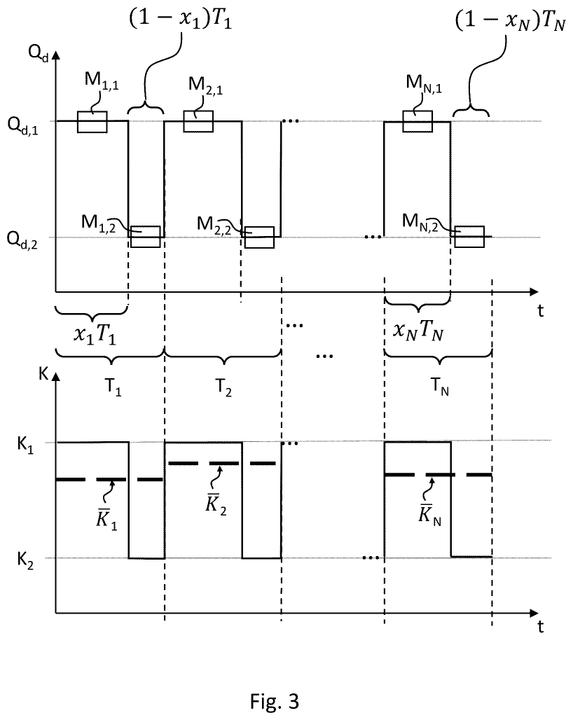

FIG. 3 shows the temporal course of a clearance-controlled change of the dialysate flow during the treatment of a patient Pa by a blood treatment apparatus 100, comprising a control device or closed-loop control device 150, in a further embodiment.

Reference is made to the reference numerals in the preceding figures.

For the sake of simplicity, the following descriptions with regard to FIG. 3 are based on a 1-pool model, e.g., the internal exchange between intravascular space (vascular system), interstitial space (intercellular space) and intracellular space has not been considered here. When calculating the dialysis dose Kt/V, it is assumed in these models that immediately after the dialysis of patient Pa, the urea concentration in their entire body is equally low, but this is not the case in the real patient Pa (see also the explanations with regard to the 2-pool model detailed herein).

The removal of a substance not contained in the dialysis liquid with concentration c, which is uniformly distributed in the patient in a substance-specific distribution volume V by a purification procedure with clearance K can be described in the 1-pool model as follows:

$$d ( Vc ) = - Kc ( t ) dt \qquad ( Formula 1 )$$

With constant K and V, the time-dependent solution for the concentration is:

$$c ( t ) = {c}_{0} {e}^{- \frac{K}{V} t} \qquad ( Formula 2 )$$

If K changes during the course of treatment, the entire treatment may be divided into N successive sections with duration Tⱼ and associated clearance Kⱼ. With

$$t = {\sum }_{j = 1}^{N} {T}_{j} ,$$

it applies for the concertation at point of time t:

$$c ( t ) = {c}_{0} {\prod }_{j = 1}^{N} {e}^{- \frac{{K}_{j}}{V} {t}_{j}} = {c}_{0} {e}^{- {\sum }_{j} \frac{{K}_{j}}{V} {t}_{j}} \qquad ( Formula 3 )$$

Thus, with constant volume of distribution V, the final concentration c(t) depends only on the sum ΣⱼKⱼtⱼ, so that instead, also the clearance averaged over the previous treatment duration T_{dial}

$$〈 K 〉 = \frac{1}{{T}_{dial}} {\sum }_{j} {K}_{j} {t}_{j} = \frac{1}{{T}_{dial}} {\int }_{{T}_{dial}} K ( t ) d t \qquad ( Formula 4 )$$

can be used:

$$c ( {T}_{di al} ) = {c}_{0} {e}^{- \frac{〈 K 〉}{V} {T}_{dial}} \qquad ( Formula 5 )$$

The temporal course of K is, therefore, irrelevant for the end concentration.

Kⱼ can depend on dialysis flow Q_{d,j}, wherein K(Q_{d}=0)=0.

In addition to one or more different values for the dialysate flow Q_{d}, the dialysate flow Q_{d} through the dialyzer 303 can be set to zero with each blood treatment apparatus by switching off the dialysis liquid production or by temporarily diverting the dialysis liquid past the dialyzer 303 (bypass). During on-line HDF procedures, in which the on-line produced dialysis liquid flows through both dialyzer 303 for diffusive substance exchange and is used as substitute fluid by diverting a partial flow, the dialysate flow Q_{d} through the dialyzer 303 may therethrough be reduced by increasing the portion used for substitution.

By switching between two values Q_{d,1} and Q_{d,2} within a time interval Tⱼ, there may be generated, by selecting the time fraction

$$x = \frac{{T}_{1}}{{T}_{j}} ,$$

wherein T₁ denotes the duration during which the dialysate flow is set with Q_{d,1}, and vice versa, any averaged clearance K̅ between K₁ and K₂:

$$\overline{K} = x {K}_{1} + ( 1 - x ) {K}_{2} \qquad ( Formula 6 )$$

For Q_{d,2}=0 (i.e., dialysate flow off) with K₂=0 is, thus, K̅=xK₁.

For quasi-continuous operation, the entire treatment may be divided into N successive sections j with duration Tⱼ and a predetermined average clearance K̅ⱼ which is to be achieved over the duration Tⱼ. K̅ⱼ is realized by switching according to Formula 1, e.g., in the simplest case by switching off the flow through the dialysis liquid chamber 303a of the dialyzer 303 after the time duration xⱼTⱼ, which can also be referred to here as point of time tⱼ.

Knowledge of K₁ and K₂ is required to determine x. These may be estimated from a model of the dialyzer 303 as a function of the dialyzer parameter K₀A and the flows.

Models are described in the literature (e.g., Sargent & Gotch, “Principles and Biophysics of Dialysis” in “Replacement of renal function by dialysis”) that can be used to calculate clearance K given knowledge of the dialyzer parameter K₀A and the flows at the dialyzer 303.

$${D}_{diff} = {Q}_{Bi} \frac{{e}^{γ} - 1}{{e}^{γ} - \frac{{Q}_{b}}{{Q}_{d}}} , \qquad ( Formula 7 )$$

$$γ = {k}_{0} A \frac{{Q}_{d} - {Q}_{Bi}}{{Q}_{Bi} {Q}_{d}}$$

In this,

D_{diff} denotes the diffusive fraction of clearance K in the dialyzer 303 Q_{Bi} denotes the total flow on the blood-side

The following applies

| | |
|---|---|
| Q_{Bi} = Q_{b} | for treatments with HD and HDF postdilution; |
| Q_{Bi} = Q_{b} + Qₛ | for treatments with HDF predilution, wherein |
| | Qₛ is the substitution rate. |

The total expected clearance is then calculated taking into account the dialysis method used

$${K}_{ref} = \frac{{Q}_{b}}{{Q}_{b} + {κ Q}_{s}} ( {D}_{diff} \frac{{Q}_{b} - {Q}_{f} - ( 1 - κ ) {Q}_{s}}{{Q}_{b} + κ {Q}_{s}} + {Q}_{f} + {Q}_{s} ) \qquad ( Formula 8 )$$

$$κ = \{ \begin{matrix}0 & HD , HDF - post \\ 1 & HDF - pre\end{matrix}$$

wherein Q_{f} denotes the net ultrafiltration rate, i.e., the liquid removal by ultrafiltration (in units [ml/min]).

In this, it should be taken into account that an effective value (K₀A)_{eff} of the dialyzer parameter must be used for K₀A, which differs substantially from the manufacturer's specifications derived from laboratory measurements (e.g., Depner “Dialyzer Performance in the HEMO Study: In Vivo K₀A and True Blood Flow Determined from a Model of Cross-Dialyzer Urea Extraction”, ASAIO Journal 2004) and takes into account the real blood characteristics and the properties of the blood circuit.

On the other hand, it is however also possible to set K₁ and K₂ during treatment by measurements on the blood-side or dialysate-side in order to be able to react, if necessary, to changing conditions during treatment.

The following results from Formula 6

$$x = \frac{\overline{K} - {K}_{2}}{{K}_{1} - {K}_{2}} \qquad ( Formula 9 )$$

Advantageously, the interval duration Tⱼ is selected such that a measurement of K₁, is possible, and with Q_{d,2}>0, additionally a measurement of K₂. It is also possible to measure only K₂ or K₁ within the individual intervals and then to use the measured value in the following interval, or to adjust the length of the intervals such that a longer interval with clearance measurement is followed by a shorter interval without clearance measurement.

At the beginning of the treatment, a mean clearance of K̅₁ is to be achieved for the duration T₁. In this, the blood treatment apparatus 100 has two discrete dialysate flow settings Q_{d,1} and Q_{d,2}, wherein Q_{d,2} may be zero.

The treatment begins with an initial dialysate flow Q_{d,1}.

Based on the knowledge of the blood flow and an estimate of the effective dialyzer parameter (K₀A)_{eff}, estimates of the clearances K₁ and K₂ may now be made according to Formula 3, where, e.g., K₂=0. From this, the time fraction x₁ may now be determined according to Formula 4, i.e., the time fraction of the interval T₁ in which the dialysate flow Q_{d,1} should be set. Advantageously, a measurement M_{1,1} is performed as soon as possible to set K₁ and a subsequent determination of K₁ as shown in FIG. 2 in order to be able to set a more precise (target) value for x₁.

After the end of this measurement M_{1,1} for setting K₁, a decision is in some embodiments made concerning the further procedure in the interval T₁: If Q_{d,2}=K₂=0, then the treatment is continued with dialysate flow Q_{d,1} until the time period x₁T₁ has elapsed, subsequently Q_{d}=Q_{d,2}=0 is set. In the event that the measurement has lasted so long that the x>1, T₁ is then prolonged so that after the expiration of the now prolonged T₁, the mean clearance K̅₁ has been administered hereby. In this event, the duration of the following time interval as well as the specification for the mean clearance to be achieved in the following time interval may be adjusted. If Q_{d,2} and thus K₂>0, the dialysate flow Q_{d,2} is set immediately after the end of the measurement of K₁ and a measurement M_{2,2} is performed with subsequent determination of K₂. As for the case of Q_{d,2}=K₂=0, a decision is now made about an extension of T₁ with compensation in the following interval. In the event that K₁>K̅₁, then Q_{d,2}=0 is set.

In the following intervals, the clearances at Q_{d,1} and Q_{d,2} determined in the first interval are now taken as the basis for the calculation of x, since it can be assumed that at constant flow ratios the clearance changes only slightly.

A separate default value for the mean clearance K̅_{N} may exist for each time interval. In the individual intervals, however, these values may be updated by further measurements with subsequent determination of the clearance, wherein only one of the values or both values may be determined respectively.

This procedure makes it possible to set any small values of the diffusive clearance in a controlled manner.

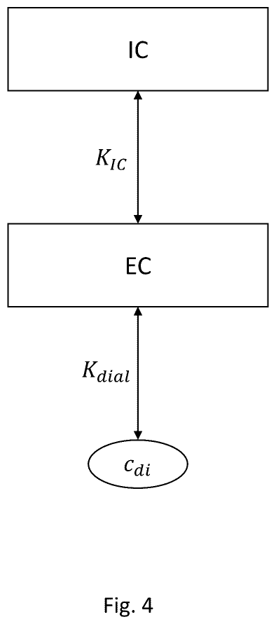

FIG. 4 shows a kinetic 2-pool model for substance exchange between body compartments IC and EC.

The 1-pool model considered so far cannot explain the effect observed in the clinic that the concentration of substances, e.g., urea, in the blood increases again immediately after the end of the dialysis. This effect is called “rebound” and can be explained by a 2-pool model (see e.g., Gotch, “Replacement of Renal Function by Dialysis”), in which the internal exchange between intercellular space, which includes the intravascular space (vascular system), and intercellular space is taken into account.

Only the extracellular space EC is accessible for dialysis, e.g., a substance exchange must take place via the internal clearance K_{IC} with the intracellular space IC. This substance exchange continues even after the end of dialysis until the concentration in all compartments EC and IC has equalized to the value c_{eq}.

The model in FIG. 4 shows the intercellular and intracellular space IC, which comprises a volume V_{IC} and a substance concentration c_{IC}. By the internal clearance K_{IC}, the substance concentration c_{IC} in the intercellular space IC and the substance concentration c_{EC} of the extracellular space EC, which in turn comprises a volume V_{EC}, are adjusted according to the principle of diffusion.

By the clearance K_{dial}, which takes place during the treatment session, substance removal from the patient's blood takes place during the treatment session depending on the concentration c_{di}.

$$d ( {V}_{IC} {c}_{IC} ) = - {K}_{IC} ( {c}_{IC} ( t ) - {c}_{EC} ( t ) ) dt \qquad ( Formula 10 )$$

$$d ( {V}_{EC} {c}_{EC} ) = - {K}_{dial} {c}_{EC} ( t ) dt$$

The dialysis dose Kt/V may be calculated after rearranging Formula 5 from the ratio of the blood-side substance concentration at the beginning of treatment c₀ and the final concentration c_{end}, where in the 1-pool model K corresponds to the mean clearance [symbol] K [symbol] during dialysis:

$$\frac{K t}{V} = - ln ( \frac{{c}_{0}}{{c}_{end}} ) \qquad ( Formula 11 )$$

However, since the patient shows a 2-pool behavior, the concentration of substances not contained in the dialysis liquid increases again in the blood after the end of the treatment session, so that c_{eq}>c_{end}. Therefore, the dialysis dose (Kt/V)_{eg} calculated from the equilibrated concentration is always lower than the value from the 1-pool model (Kt/V)_{SP}, which corresponds in good approximation to the value obtained by continuous measurement on the device side and determination of clearance based on it.

For clinical practice, methods for conversion between (Kt/V)_{eq} and (Kt/V)ₛₚ are offered (see, e.g., Daugirdas “Solute Solver,” available on http://www.ureakinetics.org/).

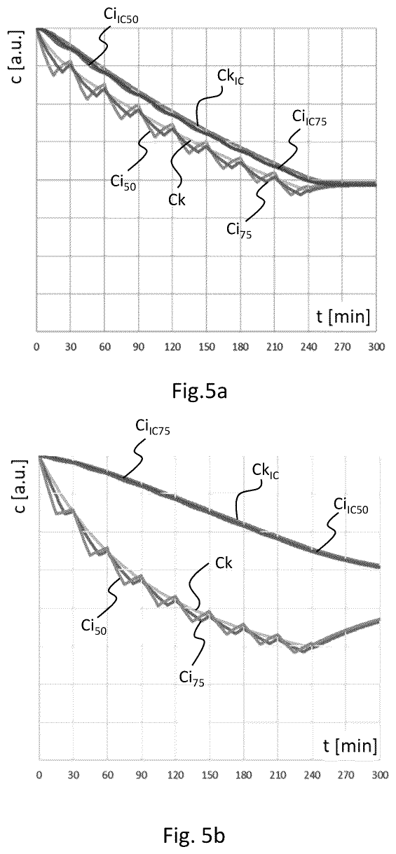

The equilibrated dialysis dose is not influenced by the intermittent procedure disclosed herein, so that the conversion models used are still valid (see FIG. 5a and FIG. 5b).

FIG. 5a shows a numerical simulation of the substance concentration course of a substance such as phosphate assuming a high intercompartmental clearance of 800 ml/min.

By numerical simulations it can be shown, for example, that (Kt/V)_{eq}=( [symbol] K [symbol] t/V)_{eq} ( [symbol] K [symbol] : mean clearance according to formula 4) is independent of x and T to a good approximation.

FIG. 5a shows the concentration curve Ck for continuous clearance behind the concentration curve Ci₅₀ in an intermittent clearance with a time fraction x=50% and the concentration curve Ci₇₅ at an intermittent clearance with a time fraction x=75%. These are the concentration courses in the intravascular space EC (vascular system) of the patient, which is directly accessible to dialysis.

Above these curves in FIG. 5a, there are the concentration curve Ck_{IC} at continuous clearance behind the concentration curve Cl_{IC50} at intermittent clearance with a time fraction x=50% and the concentration curve Ci_{IC75} in an intermittent clearance with a time fraction x=75%. These are the concentration courses in the intercellular and intracellular space IC.

In the simulation, a periodic change between a state with dialysate flow Q_{d,1} and without dialysate flow (Q_{d,2}=0) was assumed such that [symbol] K [symbol] was the same in all cases.

The intermittent course of “cleaning” the intravascular space EC shows up in the recurrent increase in concentration at times without dialysate flow (Q_{d,2}=0).

Overall, it can be seen that the concentration course in the intermittent procedures does not differ significantly from the concentration course at continuous clearance, so that, measured against each other, the concentrations are almost identical at the end of the treatment session by or after rebound.

Thus, the conversion methods developed for the continuous case between (Kt/V)_{eg} and (Kt/V)ₛₚ are still applicable.

FIG. 5b shows a numerical simulation of the substance concentration course of a substance such as phosphate analogous to FIG. 5a assuming a low intercompartmental clearance of 100 ml/min.

Reference is made to the statements with regard to FIG. 5a.

It can be seen that the concentration course for the intermittent procedure does not deviate significantly from the concentration course at continuous clearance, so that here too after the end of dialysis the concentrations are almost identical in the rebound.

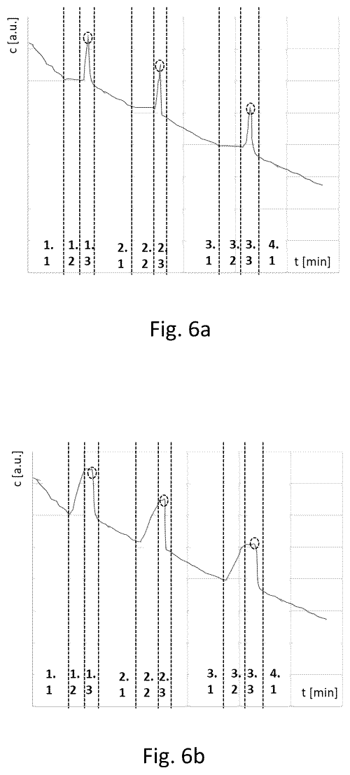

FIG. 6a shows a possible concentration course on the dialysate-side downstream of the blood filter 303 during intermittent dialysate flow without fluid removal by ultrafiltration.

Reference is made to the reference numerals and statements with regard to the preceding figures.

In the graph, the concentration c is shown in arbitrary unit [a.u.] over the time t in [min].

In the following, the case is considered in which there is an alternation between only one active dialysate flow Q_{d,1} and a switched-off dialysate flow (Q_{d,2}=0).

When the dialysate flow Q_{d,1} is active, the substance concentration on the blood-side (index “bi”) may be set from the concentrations on the dialysate-side (index “di”: dialysis liquid inlet line, “do”: dialysate outlet line) (see e.g., Sargent & Gotch, “Principles and biophysics of dialysis” in “Replacement of renal function by dialysis”)

$${c}_{bi} = {c}_{di} + \frac{{Q}_{d}}{K} ( {c}_{do} - {c}_{di} ) \qquad ( Formula 12 )$$

At the same time, it may be seen that no measurement is possible for Q_{d,2}=0 and simultaneous K₂=0. However, in a first approximation (1-pool model), it can be assumed that the substance concentration does not change when K₂=0. Therefore, for the times with Q_{d,2}=0, instead of a pause on the display, the value last determined with active dialysate flow Q_{d,1} may continuously be displayed, for example on the display device 500. This applies, e.g., to the plasma sodium.

For cases in which there is a high concentration difference between blood and dialysis liquid or dialysate, respectively, the concentration course may alternatively be numerically approximated and displayed in the 2-pool model according to Formula 10.

One possibility for determining the clearance K is the spectroscopic measurement of the concentration course of a marker substance in the spent dialysate, e.g., in the dialysate outlet line, or of a value correlating with it (e.g., “Adimea”, BBraun) using a sensor 400a and Formula 11.

When the dialysate flow Q_{d,1} is running, the substance concentration in the outflowing dialysate is always lower than in the blood. If the flow through dialyzer 303 is switched off, the substance concentration of the dialysate still present in the dialyzer 303 increases by diffusion to the substance concentration in the patient's blood. If the dialysate flow through sensor 400a is stopped completely, the concentration there does not change; if, on the other hand, only the dialysate flow through dialyzer 303 is stopped and instead the fresh dialysis liquid is fed directly to sensor 400a in a bypass circuit, the concentration there drops to zero or to the concentration in the fresh dialysis liquid.

If the dialysate flow Q_{d} is now switched on again, the dialysate with the increased substance concentration flows to the sensor 400a. With complete equilibration during the downtime, the concentration on the dialysate-side is then, at the sensor, briefly equal to the concentration on the blood-side. This is shown schematically in FIG. 6a.

In phases 1.1, 2.1, 3.1, 4.1, the concentration on the dialysate-side decreases according to the relationship to the concentration on the blood-side described in Formula 12. In these phases, determining the dialysis dose Kt/V is possible.

The concentration on the dialysate-side no longer changes in phases 1.2, 2.2, 3.2, when the dialysate flow is switched off (Q_{d,2}=0) without fluid removal by ultrafiltration. In this phase, very precise measurements of the substance concentration are possible, since a measurement period of several minutes is available.

In phases 1.3, 2.3, 3.3, the concentration on the dialysate-side briefly increases maximally to the concentration on the blood-side. The concentration on the blood-side may, therefore, be taken directly from the peak concentration and be displayed. On the other hand, determining the dialysis dose Kt/V is not possible in this range, since an increase in concentration would correspond to a negative clearance.

For this reason, the method for measuring Kt/V by concentration measurement can be supplemented by a method which, while knowing time and duration of the downtime of the dialysate flow, excludes phases j.2 and j.3 (j=1, . . . , N) from the calculation and only continues again when equilibrium between the substance concentration in blood and dialysate was restored in phase j.1. This may be the case, for example, after a dialysate flow-dependent waiting time.

FIG. 6b shows a possible concentration course on the dialysate-side downstream of the blood filter 303 during intermittent dialysate flow with fluid removal by ultrafiltration.

Reference is made to the reference numerals and statements with regard to the preceding figures, in particular with regard to FIG. 6a.

If liquid is removed by ultrafiltration during the downtime of the dialysate flow (Q_{d,2}=0), then, if the UF volume flow is sufficiently high, the dialysate at the sensor 400a is displaced by the ultrafiltrate, so that the same concentration is finally present here as in the blood, which may thus be measured directly. The volume removal required for this corresponds approximately to the volume of the dialyzer 303 on the dialysate-side, e.g., approximately 100 ml. Therefore, if this method is to be used for measurement on the dialysate-side of concentration on the blood-side, it is advantageous to increase the ultrafiltration rate during the period of dialysate downtime. For example, it may be advantageous to turn off ultrafiltration completely during the phase with dialysate flow Q_{d,1} and make up for it during the period without dialysate flow, so that the overall mean UF rate remains unchanged. Information concerning the volume of the dialyzer may be obtained by input, for example by using the communication device 600, or by measurements on the device side, e.g., the time span of an upstream given change in conductivity until reaching sensor 400a. This allows the system to decide whether a sufficiently high ultrafiltration volume has been conveyed and, thus, whether a reliable determining of the concentration on the blood-side is possible.

The method described herein for measuring substance concentrations can also be used in the case when the substance to be set is already present in the fresh dialysis liquid. For example, this applies to the measurement of electrolyte concentrations, here, e.g., of sodium, by conductivity cells or ion-selective electrodes. Here, the peak concentrations in phases 1.3, 2.3, 3.3 without liquid removal (see FIG. 6a) or in the event of liquid removal by ultrafiltration, result in the concentrations at the end of phases 1.3, 2.3, 3.3, the concentrations which are in the so-called Donnan equilibrium with the concentration on the blood-side in the plasma water. For cations like sodium the following applies:

$${c}_{bi , plw} = \frac{1}{α} {c}_{di} \qquad ( Formula 13 )$$

α is the Donnan factor. This depends on the protein concentration in the blood and is typically 0.95. If instead, which is more in line with medical practice, the concentration is to be given in relation to the total plasma, the volume of the proteins contained in the plasma can be taken into account, which is approx. 5%. In this case the following applies:

$${c}_{bi , total pl .} \approx {c}_{di} \qquad ( Formula 14 )$$

In contrast, the anion concentration in the plasma water and in the plasma is reduced, so that the following applies here:

$${c}_{bi , plw} = {α c}_{di} \qquad ( Formula 15 )$$

$${c}_{bi , total pl .} \approx 0.95 α {c}_{di}$$

The time during dialysis is also often used to administer medication, e.g., iron or vitamin preparations. When used for acute dialysis, a wide range of other medications (e.g., painkillers, antibiotics, steroids, etc.) are also administered. Here, it is advantageous to synchronize these drug administrations to phases without dialysate flow in order to avoid dialyzing out the administered substances prior to completely distributing it in the patient.

Further, in this regard, it is possible to effect the addition in the arterial branch of the system, rather than in the venous branch as is common practice, to avoid immediate dialyzing out on the way through the dialyzer 303.

Thus, the addition can be carried out on the negative pressure side of the extracorporeal system, allowing, in certain embodiments, devices in which the withdrawal from a reservoir containing the drug to be added is carried out solely by the arterial negative pressure of the extracorporeal system.

The mean clearance K̅ to be achieved in the individual time intervals may be specified on the basis of patient-specific or general empirical values or based on kinetic models for the change in substance concentration in the various body compartments.

For example, it may be known for a patient that the disequilibrium syndrome only occurs when the diffusive clearance exceeds a certain threshold, e.g., 100 ml/min, in the first 30 min of dialysis. Also, this threshold, set based on clinical experience, may depend on the day of the week and may differ from that of other days, e.g., on the day after the long dialysis-free interval (e.g., Monday or Tuesday). This or similar information may be entered via the communication device 600 or provided to the control device or closed-loop control device to be used in the calculations.

Alternatively, the time-dependent concentration difference between the compartments and thus, for example, the osmotic pressure may be calculated based on measurements or estimates of the initial substance concentration in the blood and on the assumption of an initial equilibration between the body compartments, with the aid of multi-compartment models, analogous to those explained herein, for example, as 2-pool model, which optionally contain the brain volume as a further compartment. The target or desired clearance of the dialysis system may then be calculated such that a critical concentration difference or a critical osmotic pressure is not exceeded. Above all, this applies with molecules such as urea or ethanol.

Alternatively or additionally, the limiting value may be a critical rate of change of the substance concentration. With respect to the plasma sodium, e.g., empirical values exist here from the intensive care unit (e.g., daily change <6 mmol/L). Here, the following applies in accordance with Formula 1 and Formula 10 with the sodium concentration c_{dial} in the fresh dialysate:

$$d ( V {c}_{Pat} ) = - {K}_{dial} ( {c}_{Pat} ( t ) - {c}_{dial} ) dt \qquad ( Formula 16 )$$

With a fixed volume of distribution V, the following applies for the temporal change in the concentration in the patient

$$Δ {c}_{Pat} ( t ) = ( {c}_{dial} - {c}_{Pat} ( 0 ) ) ( 1 - {e}^{\frac{K}{V} t} ) \qquad ( Formula 17 )$$

If Δc_{Pat} is to remain limited to the value Δc_{Patmax} within the time tₘₐₓ at a given sodium concentration in the dialysate c_{dial}, the clearance must remain limited to the value Kₘₐₓ:

$${K}_{max} < - \frac{V}{{t}_{max}} ln ( 1 - \frac{Δ {c}_{Pat , max}}{{c}_{dial} - {c}_{Pat} ( 0 )} ) \qquad ( Formula 18 )$$

This should be explained using the following example:

The sodium concentration in the blood of a hyponatremic patient with V=40 L and a plasma sodium of C_{Pat}(0)=120 mmol/L is to be returned to the sodium standard range (between 135 mmol/L and 145 mmol/L) by an acute dialysis treatment. In this, the lowest dialysate sodium concentration that is settable on the blood treatment apparatus 100 is c_{dial}=130 mmol/L. Within the period of tₘₐₓ=10 h, the change must not be more than Δc_{Pat,max}=6 mmol/L. Hence, according to Formula 18, this results in a maximum clearance of Kₘₐₓ=61 mL/min. With the dialyzer available in the clinic and the minimum settable blood flow at the apparatus of 200 ml/min and the minimum dialysate flow of 300 ml/min, an estimation according to Formula 7 would result in a clearance of K₁=122 ml/min, which would lead to a too rapid rise in plasma sodium. By periodically switching off the dialysate flow (=>K₂=0), the time fraction x may now be calculated using Formula 9 by setting the target value of the clearance K̅=Kₘₐₓ. This value is then transferred to the control device or closed-loop control device 150. Thus, in the present case x=0.5. By measuring during the treatment and determining the clearance resulting therefrom, as described herein, x can be adjusted in the further course such that K is achieved.

### LIST OF REFERENCE NUMERALS

100 blood treatment apparatus
101 blood pump
102 dialysate outlet line
104 dialysis liquid inlet line
105 substitute fluid line
107 predilution valve
107a line associated with the predilution valve
109 postdilution valve
109a line associated with the postdilution valve
111 substitute fluid pump
131 ultrafiltration pump
150 control device or closed-loop control device
153 drainage
155 water source
157 heat exchanger
159 first flow pump
160 means for reprocessing dialysate
161 device for balancing
162 heating device
163 mixing device
163a conductivity sensor
163b conductivity sensor
165a temperature sensor
165b temperature sensor
166 concentrate supply
167 leak sensor
168 concentrate supply
169 second flow pump
171 pump, sodium pump
173 pump, bicarbonate pump
300 extracorporeal blood tube set or blood circuit
301 first line (arterial line section)
302 arterial patient tube clamp
303 blood filter or dialyzer
303a dialysis liquid chamber
303b blood chamber
303c semipermeable membrane
305 second line (venous line section)
306 venous patient tube clamp
315 Air Bubble Detector; ABD
317 single-needle chamber
318 deaeration device
325 addition site for Heparin
329 (venous) bubble trap
400a measuring device; sensor, dialysate side
400b measuring device; sensor, dialysate side
402a measuring device; sensor, blood side
402b measuring device; sensor, blood side
500 display device
600 communication device
A container; A-concentrate; sodium
B container; B-concentrate; bicarbonate
Ck concentration curve in the intercellular and intravascular space at or while continuous clearance
Ci₅₀ concentration curve in the intercellular and intravascular space with a time fraction x=50% at intermittent clearance
Ci₇₅ concentration curve in the intercellular and intravascular space with a time fraction x=75% at intermittent clearance
Ck_{IC} concentration curve in the intracellular space at continuous clearance
Ci_{IC50} concentration curve in the intracellular space with a time fraction x=50% at intermittent clearance
Ci_{IC75} concentration curve in the intracellular space with a time fraction x=75% at intermittent clearance
EC extracellular and intravascular space (vascular system) of the patient
F1 filter
F2 filter
IC intercellular and intracellular space
K clearance
K₁ first clearance
K₂ second clearance
K̅ⱼ (pre-set) mean clearance in the time interval Tⱼ (j=1, . . . , N, N∈ [symbol] )
M_{j,1} measurement at first dialysate flow Q_{d,1} in the time interval Tⱼ (j=1, . . . , N, N∈ [symbol] )
M_{j,2} measurement at second dialysate flow Q_{d,2} in the time interval Tⱼ (j=1, . . . , N, N∈ [symbol] )
Pa patient
PS1 arterial pressure sensor (optional)
PS2 arterial pressure sensor (optional)
PS3 pressure sensor (optional)
PS4 pressure sensor for measuring the filtrate pressure
PS5 pressure sensor for measuring the pressure in the dialysis liquid inlet line
Q_{b} blood flow
Q_{d} dialysate flow
Q_{d,1} first dialysate flow
Q_{d,2} second dialysate flow
t time axis
Tⱼ time interval (j=1, . . . , N, N∈ [symbol] )
V valve
V24 valve
V25 valve
VB bypass valve
xⱼ time fraction (j=1, . . . , N, N∈ [symbol] ) with first dialysate flow in the interval Tⱼ
1-xⱼ time fraction (j=1, . . . , N, N∈ [symbol] ) with second dialysate flow in the interval Tⱼ
Y Y-connector

1-13. (canceled)

14. A control device or closed-loop control device configured to control or control in a closed-loop manner an extracorporeal blood treatment apparatus, when the control device is connected to the extracorporeal blood treatment apparatus in signal communication, the extracorporeal blood treatment apparatus comprising, for extracorporeally treating the blood of a patient in a blood treatment session, a dialyzer being divided by a semipermeable membrane into a blood chamber and a dialysis liquid chamber, wherein:
the control device or closed-loop control device is further configured to set, for an extracorporeal blood treatment by the extracorporeal blood treatment apparatus, a dialysate flow (Q_{d,1}, Q_{d,2}) through the dialyzer;
the control device or closed-loop control device is configured such that the dialysate flow (Q_{d,1}, Q_{d,2}) through the dialyzer is set such that, over a plurality of successive time intervals (T₁, T₂, . . . , T_{N}, N∈ [symbol] ) within the blood treatment session, a first dialysate flow (Q_{d,1}) and a second dialysate flow (Q_{d,2}), respectively, alternate with each other, wherein the first dialysate flow (Q_{d,1}) is higher than the second dialysate flow (Q_{d,2}).

15. The control device or closed-loop control device of claim 14, comprising, or connected to, a measuring device for measuring, or to an estimating device for estimating, at least one characteristic value for a clearance (K), wherein the control device or closed-loop control device is configured such that the first dialysate flow (Q_{d,1}) and/or the second dialysate flow (Q_{d,2}) is determined based on the at least one characteristic value for the clearance K.

16. The control device or closed-loop control device of claim 14, wherein the first dialysate flow is a maximum dialysate flow settable at the blood treatment apparatus.

17. The control device or closed-loop control device according to claim 14, wherein the second dialysate flow through the dialysis liquid chamber is zero.

18. The control device or closed-loop control device of claim 14, wherein the control device or closed-loop control device is configured to set (i) a first time fraction (xⱼ) for a respective time interval (Tⱼ, j=1, . . . , N, N∈ [symbol] ) from the plurality of successive time intervals (T₁, T₂, . . . , T_{N}), during which first time fraction (xⱼ) dialysis liquid is conveyed through the dialysis liquid chamber with the first dialysate flow (Q_{d,1}) within the respective time interval (Tⱼ), and/or (ii) a second time fraction (1-xⱼ), during which dialysis liquid is conveyed through the dialysis liquid chamber with the second dialysate flow (Q_{d,2}) within the time interval (Tⱼ).

19. The control device or closed-loop control device of claim 18, wherein the first time fraction (xⱼ) and the second time fraction (1-xⱼ) are set such that the blood conveyed through the blood chamber during the time interval (Tⱼ) is purified when passing through the blood chamber with a pre-set mean clearance (K̅ⱼ).

20. The control device or closed-loop control device of claim 18, wherein the control device or closed-loop control device is configured to be in signal communication, during use, with one or several sensors of a measuring device and/or to determine clearance (K₁, K₂) from signals transmitted thereto by the sensors.

21. The control device or closed-loop control device of claim 20, wherein the control device or closed-loop control device is configured to adjust, based on values determined by the sensors, the first time fraction (xⱼ) and/or the second time fraction (1-xⱼ) such that a pre-set mean clearance (K̅ⱼ) is achieved.

22. The control device or closed-loop control device of claim 14, comprising a communication device for inputting one or more values required for setting the dialysate flow (Q_{d,1}, Q_{d,2}) for the extracorporeal blood treatment.

23. A non-transitory digital storage medium configured to interact with a programmable computer system such that a conventional control device or closed-loop control device of a blood treatment apparatus is reprogrammed into the control device or closed-loop control device of claim 14.

24. The non-transitory digital storage medium of claim 23, comprising a floppy disk, memory card, CD or DVD, EPROM, FRAM or SSD, with electronically readable control signals.

25. A computer program product, as a signal wave or having a program code stored on a non-transitory machine-readable carrier, for interacting with a programmable computer system such that a conventional control device or closed-loop control device of a blood treatment apparatus is reprogrammed into the control device of claim 14.

26. A non-transitory computer-readable medium comprising a computer program a program code configured to reprogram a conventional control device or closed-loop control device of a blood treatment apparatus into the control device of claim 14, when the computer program runs on a computer.

27. A blood treatment apparatus for an extracorporeal blood treatment, the blood treatment apparatus comprising or connected in signal communication with a control device or closed-loop control device configured to control or control in a closed-loop manner the blood treatment apparatus, wherein:
the control device or closed-loop control device is further configured to set, for an extracorporeal blood treatment by the extracorporeal blood treatment apparatus, a dialysate flow (Q_{d,1}, Q_{d,2}) through a dialyzer; and
the control device or closed-loop control device is configured such that the dialysate flow (Q_{d,1}, Q_{d,2}) through the dialyzer is set such that over a plurality of successive time intervals (T₁, T₂, . . . , T_{N}, N∈ [symbol] ) within a blood treatment session, a first dialysate flow (Q_{d,1}) and a second dialysate flow (Q_{d,2}), respectively, alternate with each other, wherein the first dialysate flow (Q_{d,1}) is higher than the second dialysate flow (Q_{d,2}).

28. The blood treatment apparatus according to claim 27, wherein:
the blood treatment apparatus comprises or is connected to the dialyzer; and
the dialyzer is divided by a semipermeable membrane into a blood chamber through which blood may flow with a pre-set blood flow (Q_{b}) and a dialysis liquid chamber through which dialysis liquid may flow with a pre-set dialysate flow (Q_{d}).

29. The blood treatment apparatus according to claim 28, wherein the second dialysate flow (Q_{d,2}) through the dialysis liquid chamber is zero.

30. The blood treatment apparatus according to claim 28, wherein the control device or closed-loop control device is configured to set (i) a first time fraction (xⱼ) for a respective time interval (Tⱼ, j=1, . . . , N, N∈ [symbol] ) from the plurality of successive time intervals (T₁, T₂, . . . , T_{N}), during which first time fraction (xⱼ) dialysis liquid is conveyed through the dialysis liquid chamber with the first dialysate flow (Q_{d,1}) within the respective time interval (Tⱼ), and/or (ii) a second time fraction (1-xⱼ), during which dialysis liquid is conveyed through the dialysis liquid chamber with the second dialysate flow (Q_{d,2}) within the time interval (Tⱼ).

31. The blood treatment apparatus according to claim 30, wherein the first time fraction (xⱼ) and the second time fraction (1-xⱼ) are set such that the blood conveyed through the blood chamber during the time interval (Tⱼ) is purified when passing through the blood chamber with a pre-set mean clearance (K̅ⱼ).

32. The blood treatment apparatus according to claim 27, wherein:
the blood treatment apparatus comprises or is connected to (i) a measuring device for measuring at least one characteristic value for a clearance (K) or (ii) an estimating device for estimating the at least one characteristic value for a clearance (K); and
the control device or closed-loop control device is configured such that the first dialysate flow (Q_{d,1}) and/or the second dialysate flow (Q_{d,2}) is determined on a basis of the at least one characteristic value for the clearance.

33. The blood treatment apparatus according to claim 27, wherein the first dialysate flow is a maximum dialysate flow settable at the blood treatment apparatus.
